(19) United States
(12) Patent Application Publication
(10) Pub. No.: US 2006/0154302 A1
(43) Pub. Date: Jul. 13, 2006

(54) Iterative, subtractive immunoaffinity method for proteome analyte enrichment

(76) Inventor: Thomas R. Brown, Rochester Hills, MI (US)

Correspondence Address: Ronald W. Citkowski;Gifford, Krass, Groh, Sprinkle,, Anderson & Citkowski, P.C., PO Box 7021, Troy, MI, 48007-7021, US

(21) Appl. No.: 11/331,382

(22) Filed: Jan. 12, 2006

Related U.S. Application Data
(60) Provisional application No. 60/643,379, filed on Jan. 12, 2005.

(51) Int. Cl. G01N 33/53; G01N 33/558

(52) U.S. Cl. 435/7.1; 436/514

(57) ABSTRACT

An immunoaffinity method according an embodiment of the present invention is described for selectably removing highly abundant proteins (HAP) from a sample which contains a mixture of HAP and lower abundance proteins (LAP). One method includes providing a sample having a concentration of original HAP and LAP therein and producing antibodies to the sample. The sample is subjected to a subtractive immunoaffinity chromatography process which employs the antibodies produced. A purified sample is collected which has been subjected to the subtractive immunoaffinity chromatography process. The purified sample is characterized in that the concentration of original HAP has been selectably reduced as compared to the concentration of original LAP. The purified sample now includes a concentration of new HAP and LAP. Also described are antibody compositions, methods for purification of a specimen for analysis, and systems for carrying out the described methods.

### REFERENCE TO RELATED APPLICATION

This application claims priority from U.S. Provisional Patent Application Ser. No. 60/643,379, filed Jan. 12, 2005, the entire content of which is incorporated herein by reference.

### GOVERNMENT SPONSORSHIP

This work was supported in part by Grant No. NIH-2R44-ES012277. Accordingly the United States government may have certain rights in this invention.

### FIELD OF THE INVENTION

This invention relates generally to assay methods for proteins in biological materials. More specifically, the invention relates to a sample preparation method for selectively enhancing the content of low abundance proteins in samples used for proteomic analysis.

### BACKGROUND OF THE INVENTION

Analysis of the proteome of various organisms, including humans, is a very active area of research. Proteomic analysis is complicated by the fact that numerous proteomes include a very large number of different proteins, and the concentration of these proteins usually varies over many orders of magnitude. Many, if not most, of the proteins of most interest are present in relatively low amounts. These proteins are generally referred to as low abundance proteins (LAP). Their presence is often masked by high abundance proteins (HAP) that, in serum, include albumin, immunoglobulins and other proteins. In order to perform a proteomic analysis on protein-containing biofluids, it is advantageous to separate the HAPs from the LAPs.

Heretofore the prior art has attempted to remove specific HAPs by the use of protein-binding chemicals as well as monoclonal and polyclonal antibodies which are specific for particular proteins. In particular instances, cocktails of antibodies, each of which is specific for a generally encountered HAP, have been utilized for sample preparation. Such methods are expensive and constrained since, in many instances, the cocktails contain antibodies to only a few of the HAPs.

While the foregoing prior art method can target specific proteins, there is no method known in the prior art which can target and remove proteins on the basis of their abundance. This fact is reflected in a publication by Lathrop, J. T. et al. in Current Opinion in Molecular Therapeutics 5(3):250-257 (2003) wherein it was stated: “Proteins can be separated by charge, size, and isoelectric point, but there are currently no methods to separate proteins by abundance.”

As will be detailed hereinbelow, the present invention is directed to a method which targets and removes proteins from a sample on the basis of their abundance. The method of the present invention specifically targets HAPs in a protein-containing sample, and operates to selectably remove at least a portion of the HAPs thereby enhancing the concentration of LAPs in that sample.

### SUMMARY OF THE INVENTION

An immunoaffinity method according an embodiment of the present invention is described for selectably removing highly abundant proteins (HAP) from a sample which contains a mixture of HAP and lower abundance proteins (LAP). Such an embodiment includes providing a sample having a concentration of original HAP and LAP therein and producing antibodies to the sample. The sample is subjected to a subtractive immunoaffinity chromatography process which employs the antibodies produced. A purified sample is collected which has been subjected to the subtractive immunoaffinity chromatography process. The purified sample is characterized in that the concentration of original HAP has been selectably reduced compared to the concentration of original HAP in the unpurified sample. Further, the purified sample is enriched in original LAP compared to the concentration of original LAP in the unpurified sample. The purified sample now includes a concentration of new HAP and LAP.

In one embodiment, a further iteration of antibody production and a subtractive immunoaffinity chromatography process is included. Thus, in such an embodiment, a purified sample, that is, a sample subjected to a subtractive immunoaffinity chromatography process to reduce original HAP, is provided. Antibodies to the purified sample are produced and used to subject the purified sample to a subtractive immunoaffinity chromatography process. A second purified sample is thus produced. The new HAP are selectively reduced in the second purified sample.

In further embodiments, at least one additional iteration of producing antibodies to at least one further purified sample is included in an inventive method. Also included in further embodiments is subjecting the at least one further purified sample to at least one further subtractive immunoaffinity chromatography process using the produced antibodies. As many further iterative steps of sample purification and producing antibodies to the further purified samples may be performed as desired.

A composition is provided according to an embodiment of the present invention which includes a plurality of antibodies raised to a sample. In a preferred embodiment, an inventive composition includes a plurality of antibodies raised to a purified sample, wherein the purified sample has a reduced concentration of HAP compared to a concentration of original HAP present in an unpurified sample. Optionally, an inventive composition includes a plurality of antibodies raised to an unpurified sample. In a further option, an inventive composition includes a plurality of antibodies raised to an unpurified sample and one or more pluralities of antibodies raised to one or more iteratively purified samples.

A method for purifying a specimen is described according to an embodiment of the present invention which includes providing a first plurality of antibodies raised to an unpurified sample. In a preferred embodiment, this first plurality of antibodies is attached to a support. Optionally, a second plurality of antibodies raised to a purified sample is provided, the second plurality of antibodies also preferably attached to a support.

The first plurality of antibodies and/or the second plurality of antibodies are brought into contact with a specimen which includes a mixture of HAP and LAP, under binding conditions, such that at least a portion of the HAP in the specimen bind to the first and/or second pluralities of antibodies. The specimen is eluted away from the bound HAP, thus producing a purified specimen since binding and removal of at least a portion of HAP results in a concentration of HAP in the specimen being selectively reduced as compared to an original concentration of HAP in an unpurified specimen. Further, the specimen is enriched in LAP.

As many further iterative steps of specimen purification may be performed as desired.

A system for preparing a specimen for analysis is provided which includes a first plurality of antibodies raised to a sample which contains a mixture of HAP and LAP. The first plurality of antibodies are preferably attached to a support.

Optionally further included is a second plurality of antibodies raised to a sample. The second plurality of antibodies is preferably attached to a support.

In one embodiment, the first plurality of antibodies includes antibodies raised to an unpurified sample. The first plurality of antibodies may alternatively include antibodies raised to a purified sample.

Optionally, one or more pluralities of antibodies raised to one or more iteratively purified samples may also be included.

A support to which the antibodies are optionally attached preferably includes a material such as a natural or synthetic polymer, resin, silicate, or a combination thereof. Particular illustrative examples of suitable materials include an agarose; a cellulose, illustratively including a carboxymethyl cellulose; a cellulose acetate; a dextran; a divinylbenzene; a methacrylate; a polymethacrylate; glass; a ceramic; a paper; a metal; a metalloid; a nitrocellulose; or a nylon; a polyacryloylmorpholide; a polyamide; a poly(tetrafluoroethylene); a polyethylene; a polypropylene; poly(4-methylbutene); a poly(ethylene terephthalate); a polyformaldehyde; a polyacrylamide; a polystyrene; a polyethylene glycol; a rayon; a poly(vinyl butyrate); a polyvinylidene difluoride (PVDF); a resin; a silicone; a silicate; or a combination of such materials.

A support is preferably provided in a form such as a membrane, a surface, beads, a fine particulate, a gel, a matrix, or a combination of these.

A composition including a purified sample of protein containing material is provided according to the present invention which includes protein enriched in LAP.

### BRIEF DESCRIPTION OF THE DRAWINGS

FIG. 1 is a diagram illustrating an embodiment of an inventive for selectably removing highly abundant proteins (HAP) from a sample which contains a mixture of HAP and lower abundance proteins (LAP);

FIG. 2 is a diagram illustrating an embodiment of an inventive method for purifying a specimen;

FIG. 3 is a diagram illustrating an embodiment of an inventive method for sequential use of different pluralities of produced antibodies for purifying a specimen; and

FIG. 4 shows a 1D electrophoretic gel analysis of rat serum purified by a method according to the present invention compared to unpurified whole rat serum.

### DETAILED DESCRIPTION OF THE INVENTION

The present invention utilizes an immunoaffinity subtractive chromatography process. In the process, antibodies are raised to a sample being analyzed. As is known in the art, antibodies can be raised by immunizing an appropriate animal with the sample and collecting the resultant mixture of antibodies generated by that animal. These antibodies are then employed in a subtractive immunoaffinity chromatography process wherein the sample, serum for example, is reacted with the antibodies that are directed against it. The immune system of animals generally has a concentration threshold below which there is no antibody response, and as a consequence, the antibodies thus generated will not have significant reactivity with LAPs. Consequently, when the sample is subjected to the immunosubtractive chromatography step, HAPs highest in concentration will be selectively bound and thus removed from the sample. This leaves a sample with a new and distinct group of HAP when compared to the original sample. This process is repeated for a number of iterations with each iteration removing the next most abundant set of HAPs so as to further enhance the relative concentration of LAPs in the sample. It is notable that the present invention operates to remove proteins on the basis of their abundance only, and does not depend on any knowledge of the identity and/or nature of the proteins which are being removed.

In one embodiment, HAP include at least 90% of total protein in a sample or specimen. In further embodiments, in each successive iterative purification, HAP include at least 60-90% of total protein in each successive iterative purified sample or specimen. In still further embodiments, in each successive iterative purification, HAP include at least 90% of total protein in each successive iterative purified sample or specimen.

In a preferred embodiment, the total number of different HAP in a purified sample or specimen is typically greater than the total number of different HAP in an unpurified sample or specimen. In some embodiments, the total number different HAP in an unpurified sample or specimen is in the range between about 7-12, inclusive. In one embodiment, the total number of different HAP in a purified sample or specimen is in the range between about 8-25, inclusive. In further embodiments, the total number of different HAP in a purified sample or specimen is in the range between about 10-100, inclusive. In still further embodiments, the total number of different HAP in a purified sample or specimen is greater than 100.

Methods are provided according to the present invention for selectively reducing HAP in a sample or specimen. Such a sample or specimen is preferably a biomaterial. The term “biomaterial” is intended to refer to a material obtained from an organism which contains a mixture of HAP and LAP. In certain embodiments, biomaterial may further refer to material obtained from an in vitro source, such as cultured primary cells and cell lines. The term “biomaterial” further refers to a material purified according to a method of the present invention which contains a mixture of HAP and LAP.

The terms “sample” and “specimen” are used interchangeably to refer to biomaterial provided for antibody production and/or purification by an inventive immunoaffinity method. A sample or specimen includes a biomaterial illustratively including serum, plasma, urine, saliva, tears, breath condensate, cellular cytosol, cellular nuclei, a tissue homogenate, sweat, and combinations of any of these. In addition, the terms sample and specimen are used to refer to materials purified according to the present invention. The term “specimen” refers to the same types of biomaterials as the term “sample” and is used to emphasize that material used to produce antibodies is optionally the same or different material purified to enrich LAP for analysis of LAP.

In a preferred embodiment, a sample and a specimen are of the same type of biomaterial. For instance, both a sample and a specimen may be both be serum. Further preferred is an embodiment in which a sample and a specimen are from the same type of organism. Thus, both a sample and a specimen may be human serum. Optionally, both a sample and a specimen may be obtained from the same individual. In a further option, either or both a sample and a specimen may be an aliquot of pooled material from a single individual or multiple individuals.

Thus, for example, a sample may be obtained from a single individual animal for production of antibodies to an unpurified and/or iteratively purified sample. A specimen to be purified using the produced antibodies, such as for analysis, may be obtained from the same individual animal or from other sources such as a second individual animal, or, from a pool of samples or specimens obtained from multiple animals.

In a further example, a sample may be obtained from a single individual animal or from a pool of samples or specimens obtained from multiple animals for production of antibodies to unpurified and/or purified serum. A specimen to be purified using the antibodies, such as for analysis, may be obtained from the same individual animal or from a second individual animal, or, from the same or different pool of samples or specimens obtained from multiple animals.

Methods for selectively reducing HAP in a sample and/or specimen may be used for various purposes. For example, in one embodiment, an inventive method is used to prepare inventive compositions including antibodies raised to a sample having a reduced concentration of HAP. In addition, methods for selectively reducing HAP in a sample or specimen may be used to prepare the sample or specimen for analysis. In particular, methods are provided according to the present invention for preparing a sample or specimen for analysis of LAP.

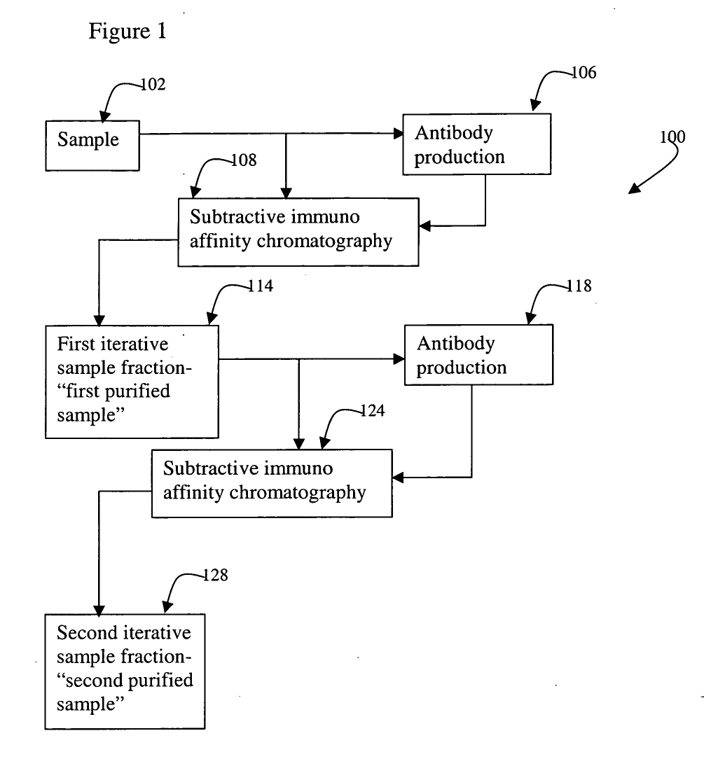

FIG. 1 illustrates an embodiment of an inventive method 100. A sample 102 is provided and used to raise a plurality of antibodies to the sample in an antibody production step 106. The sample is purified by subtractive immuno-affinity chromatography 108 using the produced antibodies to produce a first iterative sample fraction or “first purified sample” 114. In a preferred embodiment, the first purified sample 114 is used in a second antibody production step 118 to produce a second plurality antibodies to the first purified sample. The second plurality of antibodies is used in a subtractive immuno-affinity chromatography process 124 to produce a second iterative sample fraction or “second purified sample” 128.

Optionally, further iterative steps of antibody production and subtractive immuno-affinity chromatography are performed to produce further purified samples. For example, the second purified sample is used in a third antibody production step to produce a third plurality antibodies to the second purified sample. The third plurality of antibodies is used in a subtractive immuno-affinity chromatography process to produce a third iterative sample fraction or “third purified sample”.

As many further iterative steps of specimen purification may be performed as desired.

In a further option, a purified sample is collected and concentrated prior to use in producing antibodies and/or subtractive immuno-affinity chromatography process.

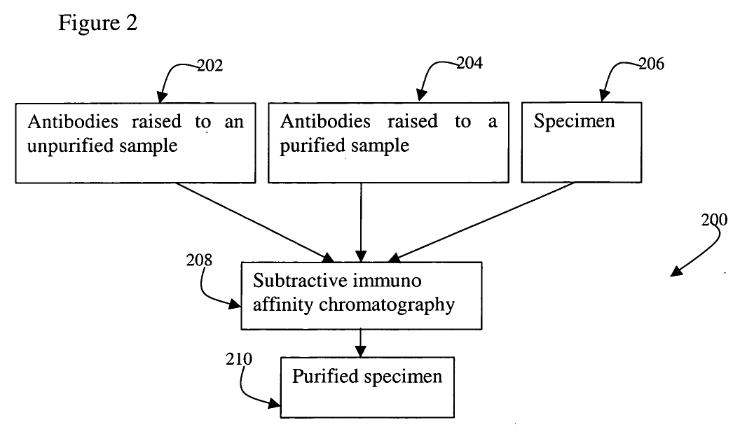

FIG. 2 illustrates an embodiment of an inventive method 200. Antibodies raised to an unpurified sample 202 and, optionally, antibodies raised to a purified sample 204 are used to purify a specimen 206 by a subtractive immunoaffinity chromatography process 208. The specimen may be purified by sequential exposure to the antibodies raised to an unpurified sample 202 and antibodies raised to a purified sample 204 in one embodiment. Alternatively, antibodies raised to an unpurified sample 202 and antibodies raised to a purified sample 204 may be exposed to the specimen in combination. In a further alternative, a specimen may be exposed to either antibodies 202 or 204 and not both.

Optionally, a purified specimen is collected and concentrated prior to being brought into contact with antibodies raised to a purified sample.

In a further option, antibodies raised to a “second purified sample” and/or antibodies raised in further iterations of antibody production and subtractive immuno-affinity chromatography are used to produce further iteratively purified samples. Such antibodies may be used sequentially or in combination to purify a specimen.

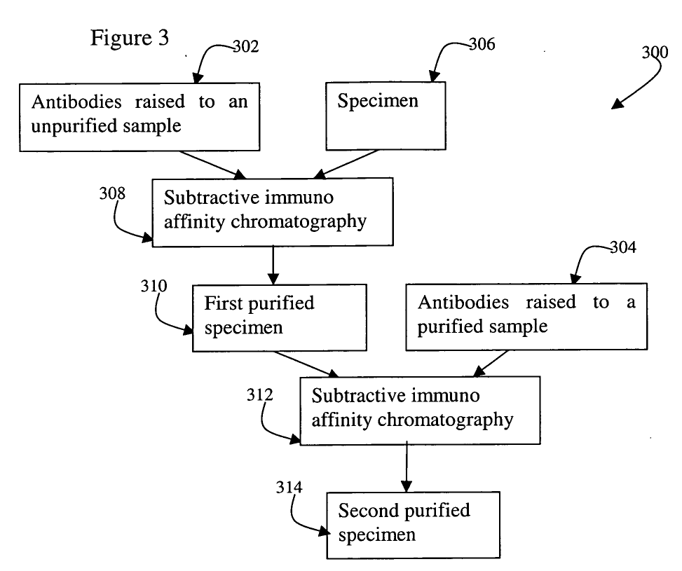

FIG. 3 illustrates an embodiment of a process according to the present invention 300 in which a specimen is sequentially exposed to antibodies. Shown is a method in which antibodies raised to an unpurified sample 302 and a specimen 306 are used in a subtractive immuno-affinity chromatography process 308 to produce a first purified specimen 310. The first purified specimen 310 and antibodies raised to a purified sample 304 are used in a subtractive immuno-affinity chromatography process 312 to produce a second purified specimen 314.

Antibodies produced, used in a method, and included in a composition and/or system according to the present invention are produced according to standard antibody production procedures. See for example procedures described in E. Harlow and D. Lane, Antibodies: A Laboratory Manual, Cold Spring Harbor Laboratory Press, 1988 and P. Shepherd and C. Dean, Monoclonal Antibodies: A Practical Approach, Oxford University Press, 2000.

Any type of antibodies may be produced for use in a method, and inclusion in a composition and/or system. In a preferred embodiment, polyclonal antibodies are produced for use in a method, and inclusion in a composition and/or system. Polyclonal antibodies are advantageous in having high affinity for antigens and in being easy to make. Further, polyclonal antibodies have a higher representation of antibodies recognizing HAP.

Antibodies produced for use in a method, and inclusion in a composition and/or system may be partially purified. For instance, an ammonium sulfate purification may be performed to produce an ammonium sulfate immunoglobulin fraction use in a method, and inclusion in a composition and/or system.

Optionally, an inventive method further includes analysis of a purified sample or specimen, a second purified sample or specimen and/or further iterative purified samples or specimens produced. Following reduction of HAP and enrichment of low abundance proteins according to a method of the present invention, a purified sample or specimen may be used in an analytical procedure. For example, the proteins may be analyzed by standard proteomics techniques such as separation by 2Dgels. Such techniques allow for recognition of differentially expressed proteins, the excision and digestion of the proteins of interest and/or their identification by any number of standard procedures.

An inventive method may be used to analyze particular LAP in certain embodiments. For example, following reduction of HAP and enrichment of low abundance protein according to a method of the present invention, a purified sample may be further purified and/or assayed using antibodies directed against a particular antigen of interest. In a particular embodiment, amino acid modifications such as nitrotyrosine or carbonyl groups that may be present on the proteins are of special interest. These newly discovered, low abundance oxidized proteins, previously unavailable due to the masking of high abundance proteins, are of interest in many clinical situations associated with oxidative stress. Thus, an embodiment of the present invention includes assays of these novel biomarkers. Such assays may be used to assess the extent of oxidative stress and/or inflammation at various stages of disease development and during therapeutic intervention.

A composition is provided according to an embodiment of the present invention which includes a plurality of antibodies raised to a purified sample. Optionally, an inventive composition further includes a plurality of antibodies raised to an unpurified sample. Further optionally, one or more further pluralities of antibodies raised to one or more further iteratively purified samples is present in an inventive composition. Such a composition may be used in preparing antibodies for use in a purification method and/or for use in purifying a specimen, such as for analysis of LAP.

A composition including a purified sample of protein containing material is provided according to the present invention which includes protein enriched in LAP and having reduced concentrations of HAP compared to LAP. Such a composition is prepared according to a method of purification described herein and may be used in analytic procedures, such as for identification and characterization of LAP.

A system for preparing a specimen for analysis is provided which includes a plurality of antibodies raised to a purified sample which contains a mixture of HAP and LAP, wherein the purified sample has a reduced concentration of HAP compared to a concentration of HAP present in an unpurified sample. The plurality of antibodies is preferably attached to a first support.

Optionally further included in an inventive system is a plurality of antibodies raised to an unpurified sample. The plurality of antibodies raised to an unpurified sample is preferably attached to a second support.

The first and second supports may be the same or different.

A ratio of different pluralities of antibodies used in an inventive method and/or present in an inventive composition is in the range between about 0.1:100-100:01, inclusive, for any two different pluralities of antibodies. In a further embodiment, the ratio of different pluralities of antibodies used in an inventive method and/or present in an inventive composition is in the range between about 0.5:50-50:0.5, inclusive, for any two different pluralities of antibodies. In a still further embodiment, the ratio of different pluralities of antibodies used in an inventive method and/or present in an inventive composition is in the range between about 1:10-10:1, inclusive, for any two different pluralities of antibodies.

In a further option, one or more further pluralities of antibodies raised to one or more further iteratively purified samples is provided and attached to further supports.

The supports to which the antibodies are optionally attached in an inventive system preferably include a material such as a natural or synthetic polymer, resin, silicate, or a combination thereof. Particular illustrative examples of suitable support materials include an agarose; a cellulose, illustratively including a carboxymethyl cellulose; a cellulose acetate; a dextran; a divinylbenzene; a methacrylate; a polymethacrylate; glass; a ceramic; a paper; a metal; a metalloid; a nitrocellulose; or a nylon; a polyacryloylmorpholide; a polyamide; a poly(tetrafluoroethylene); a polyethylene; a polypropylene; poly(4-methylbutene); a poly(ethylene terephthalate); a polyformaldehyde; a polyacrylamide; a polystyrene; a polyethylene glycol; a rayon; a poly(vinyl butyrate); a polyvinylidene difluoride (PVDF); a resin; a silicone; a silicate; or a combination of such materials. For example, commercial products sold under trade names SEPHADEX and SEPHAROSE may be used as a support in embodiments of the present invention.

A support to which antibodies are attached is preferably provided in a form such as a membrane, a surface, beads, a fine particulate, a gel, a matrix, or a combination of these. The supports for different antibodies may be the same or different type and/or form of support.

Antibodies may be attached to a support directly, or indirectly such as through a spacer arm. An antibody can be attached to a support via a reactive functional group while leaving the antibody available to bind an antigen. Illustrative examples of functional groups include alkyl, amide, amine, amino, carboxy, carbonyl, cyanate, disulfide, diazo, ether, ester, epoxides, hydroxyl, iodine, isocyanate, oxide, Si—OH, sulfhydryl, sulfonic thiocyanate, or similar groups having chemical or potential chemical reactivity.

Exemplary chemistries for attaching antibodies to agarose include cyanogen bromide (CNBr), carbonyldiimidazole (CDI), and N-hydroxysuccinimide (NHS) reagents and methods.

Further chemistries and methods for attaching antibodies to a support are known in the art and are exemplified in Hermanson, Greg T. et al., Immobilized Affinity Ligand Techniques, Academic Press, NY, 1992.

In a preferred embodiment, antibodies attached to a support are introduced into a container in an inventive system. Preferably the container allows for application of a specimen, contact between the antibodies attached to a support and the specimen, and separation of the unbound protein from the HAP bound to the antibodies.

Any suitable support and/or container may be used which allows specific binding of protein in a sample or specimen to antibodies attached to the support, and separation of the bound protein from the unbound protein. Numerous methods, materials and apparatus may be used which allow specific binding of protein in a sample or specimen with antibodies attached to the support. Further, numerous methods, materials and apparatus may be used for separation of the bound protein from the unbound protein are suitable. Illustrative methods, materials and apparatus are described in Pound, John D., Immunochemical Protocols, 2^{nd} Ed., Humana Press, NJ, 1998, for example.

An exemplary container is a disposable or reusable column. A spin column may also be used.

Thus, for example, antibodies attached to a SEPHAROSE bead resin may be poured into a column. In such a configuration, two or more pluralities of antibodies attached to supports are optionally introduced into the column sequentially, such that a specimen loaded onto the column will contact the antibodies sequentially. In a further option, a mix of pluralities of antibodies attached to supports may be present along the column.

A ratio of different pluralities of antibodies present in an inventive system is in the range between about 0.1:100-100:01, inclusive, for any two different pluralities of antibodies. In a further embodiment, the ratio of different pluralities of antibodies present in an inventive system is in the range between about 0.5:50-50:0.5, inclusive, for any two different pluralities of antibodies. In a still further embodiment, the ratio of different pluralities of antibodies present in an inventive system is in the range between about 1:10-10:1, inclusive, for any two different pluralities of antibodies.

A reduction in HAP in a sample or specimen occurs due to binding of HAP to antibodies, removing the bound proteins from the sample or specimen and thus purifying the sample or specimen. A reduction of HAP concentration in the purified sample or specimen compared to HAP concentration in an unpurified sample or specimen may be assessed by standard techniques. For example, total protein is reduced in a purified sample or specimen compared to the unpurified purified sample or specimen used as starting material. Further, reduction in HAP concentration in a purified sample or specimen compared to that in an unpurified sample or specimen may be observed by adjusting the volume of the purified and/or unpurified material to be approximately equal and analyzing the purified and unpurified materials. Such analysis may include standard techniques such as 1D and/or 2D gel analysis, immunoblot and/or immunoassay.

Methods, compositions and systems of the present invention may be used in conjunction with animals of any type, including, but not limited to, human, rat, mouse, rabbit, chicken, dog, cat, horse, pig and cow.

Embodiments of inventive compositions and methods are illustrated in the following examples. These examples are provided for illustrative purposes and are not considered limitations on the scope of inventive compositions and methods.

### EXAMPLES

### Example 1

CNBr-Activated Sepharose 4B and Ig Coupling

Protocol for CNBr activation of 200 μL Sepharose 4B.

Reagents:

Sepharose 4B from Amersham/Pharmacia.

Activation buffer: 2M sodium carbonate (Na₂CO₃)(MW=106), 212 g/L, no pH adjust needed, storing at 4° C. causes it to freeze.

Coupling buffer: 0.1M sodium bicarbonate (NaHCO₃)(MW=84), pH 8.3+0.5M NaCl (to make up 4 L, bicarb 33.6 g/4 L, NaCl 116 g/4 L). Adjust pH to 8.5. Store at 4° C.

Coupling buffer: 0.2M sodium bicarbonate (NaHCO₃)(MW=84), pH 8.3+0.5M NaCl (to make up 4 L, bicarb 67.2 g/4 L, NaCl 116 g/4 L). Adjust pH to 8.5. Store at 4° C.

Cyanogen Bromide (CNBr) from Aldrich (C91492-25G), 25 g

Acetonitrile (ACN) from Aldrich (360457-25ML), 25 mL

4 L ice cold deionized water

0.1M Tris-HCl buffer, pH 8.0, Tris-HCl 8.88 g/L, Tris base 5.30 g/L, pH to 8.0

1.0M NaCl, 58 g/L

Protein solution, see #7

Procedure

1. Divide the 1 L of Sepharose 4B into 5×200 mL aliquots and store at 4° C. Bring an aliquot to room temperature and to get ride of bubbles, swirl by hand.

2. Prepare the hood by putting down absorbent paper. Place a stop watch and two glass 10 mL pipets with a Pipet Aid (Drummond) in the hood. Stop the 4 L flask side, arm w/a rubber stopper.

3. Dissolve 25 g CNBr in 12.5 mL ACN at room temperature. Do this with gloves. It's an endothermic reaction and it takes about 30 min to get in solution

4. Wash the degassed 200 mL of resin with 500 mL high quality reagent grade water and then with 500 mL activation buffer, both at room temperature. Empty the 4 L beaker in the hood sink. Suspend the resin in 200 mL activation buffer and stir in the filter at room temperature.

5. Pipet 10 mL of the CNBr solution while stirring the resin by hand and allow the activation reaction to continue for exactly 2 min at room temperature.

6. Quickly wash the activated resin with 4 L ice cold high quality reagent grade water, empty the flask into the hood sink and discard the excess CNBr, then wash with 1 L cold coupling buffer. Use the resin immediately to couple.

7. For antisera dialyzed against coupling buffer, scoop out the resin and transfer to a 500 mL beaker containing 200 mL of coupling solution prepped as follows: couple a max of 25 mg protein/1 mL resin, q.s.'d with coupling buffer—to determine the amount of protein needed for this ratio, use the total protein calculated from the BCA assay using BgG standards. So the total volume of the stirred coupling solution containing the antiserum will be 400 mL. For antisera not dialyzed against coupling buffer, determine the amount of protein and volume of antibodies needed (as above) and add an equal volume of 0.2M Na bicarb, 0.5M NaC (16.8 g/L, 29 g/L), Then qs to 200 mL with the standard coupling buffer. Again, the total stirred volume will be 400 mL.

8. Transfer the mixture to 4° C. and using stir motor on low, stir very slowly for 16-24 hours.

9. Transfer the resin to the filter and wash the coupled resin with 2 L coupling buffer and 2 L high quality reagent grade water at room temperature to remove unreacted ligand.

10. Clamp the tubing that connects the pump to the filter flask side arm and cover the second side arm. Block excess active groups on the resin by suspending in 200 mL 0.1M Tris-HCl buffer pH 8.0 for 2 hr at room temperature Empty the 4 L flask.

11. Wash the resin with 1 L 1.0M NaCl and 1 L high quality reagent grade water at room temperature.

12. Final volume of resin is ˜150 mL. Lose 25% of the resin during the procedure. If the gel is to be used immediately, equilibrate with 0.01M PBS, pH 7.4. If not, store in PBS with 0.1% sodium azide.

### Example 2

Anti-rat serum antibodies are produced according to standard antibody production procedures. The antibodies are collected and attached to a support as described in Example 1. The resin is poured into a column and equilibrated with an appropriate buffer. A specimen of whole rat serum is subjected to purification by affinity chromatography by placing the specimen on the column under binding conditions such that HAP present in the serum bind to antibodies attached to the resin in the column. Binding conditions include such parameters as pH, temperature and salt concentration. Binding conditions for binding an antigen to an antibody in a mixture of proteins are known and one of skill in the art will recognize appropriate binding conditions without undue experimentation.

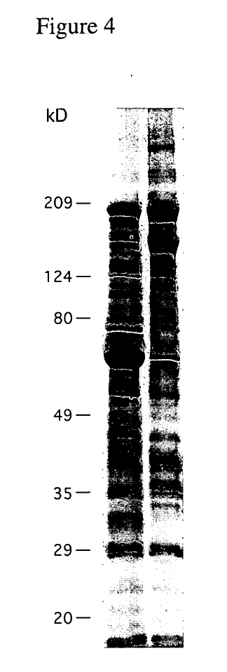

The remaining unbound serum proteins are eluted, concentrated, and analyzed. FIG. 4 shows a 1 D electrophoretic gel analysis of the purified specimen compared to unpurified whole rat serum. Approximately 90% of the total protein in the whole rat serum is removed by the subtractive immuno-affinity process and the eluted protein is concentrated about 10-fold before application to the gel. The unpurified specimen and purified specimen are separated by 5-15% gradient PAGE and stained with Coomassie blue in FIG. 4. The unpurified specimen is shown in the lane on the left while the purified specimen is shown in the right lane.

The resulting gel in FIG. 4 shows the near complete removal of albumin, at approximately 65 kD, and several additional HAP with slightly lower Mr. In addition, throughout the gels there are differences in protein concentrations and mobility, indicating the removal of HAP and the uncovering of a new population of proteins in the purified specimen.

Any patents or publications mentioned in this specification are incorporated herein by reference to the same extent as if each individual publication is specifically and individually indicated to be incorporated by reference.

The foregoing describes particular embodiments and implementations of the present invention. In view of the teaching presented herein, yet other modifications and variations thereof will be apparent to those of skill in the art. It is the following claims, including all equivalents, which define the scope of the invention.

I claim:

1. An immunoaffinity method for selectably removing highly abundant proteins (HAP) from a sample which contains a mixture of HAP and lower abundance proteins (LAP), said method comprising the steps of:
(a) providing a sample which comprises a concentration of original HAP and LAP therein;
(b) producing antibodies to said sample;
(c) subjecting the sample to a subtractive immunoaffinity chromatography process which employs the antibodies produced in step (b); and
(d) collecting a purified sample which has been subjected to the process of step (c) wherein the concentration of original HAP in said purified sample has been selectably reduced as compared to the concentration of original LAP therein and the purified sample comprises a concentration of new HAP and LAP.

2. The method of claim 1, further comprising:
(e) producing antibodies to said purified sample;
(f) subjecting the purified sample to a subtractive immunoaffinity chromatography process which employs the antibodies produced in step (e); and
(g) collecting a second purified sample which has been subjected to the process of step (f) wherein the concentration of new HAP in said purified sample has been selectably reduced as compared to the concentration of new LAP therein.

3. The method of claim 2 further comprising at least one additional iteration of producing antibodies to at least one further purified sample and subjecting the at least one further purified sample to at least one further subtractive immunoaffinity chromatography process using the produced antibodies.

4. The method of claim 1 wherein the step of producing antibodies to said sample comprises immunizing an animal with the sample of step (a), and harvesting antibodies from said animal.

5. The method of claim 1 wherein said sample is selected from the group consisting of: serum, plasma, urine, saliva, tears, breath condensate, cellular cytosol, cellular nuclei, a tissue homogenate, sweat, and combinations thereof.

6. The method of claim 1 wherein original HAP comprises at least 90% of the protein in the sample of step (a).

7. A composition comprising:
a plurality of antibodies raised to a first sample, the first sample comprising a mixture of HAP and LAP.

8. The composition of claim 7 further comprising a second plurality of antibodies raised to a second sample, the second sample comprising a mixture of HAP and LAP.

9. The composition of claim 7 wherein the first sample is an unpurified sample.

10. The composition of claim 7 wherein the first sample is a purified sample, the purified sample having a reduced concentration of HAP compared to a concentration of HAP present in an unpurified sample.

11. A method for purifying a specimen, comprising:
providing a first plurality of antibodies raised to a sample, the first plurality of antibodies attached to a first support;
contacting a specimen comprising a mixture of HAP and LAP with the first plurality of antibodies, under binding conditions, such that at least a portion of the HAP in the specimen bind to the first pluralities of antibodies and a concentration of HAP in the specimen is selectably reduced, thereby producing a purified specimen.

12. The method of claim 11 further comprising providing a second plurality of antibodies, the second plurality of antibodies raised to a second sample, the second plurality of antibodies attached to a second support; and
contacting the specimen with the second plurality of antibodies, under binding conditions, such that at least a portion of the HAP in the specimen bind to the second pluralities of antibodies and a concentration of HAP in the specimen is selectably reduced, thereby producing a purified specimen.

13. The method of claim 12 wherein the contacting the specimen with the first plurality of antibodies is performed prior to contacting the specimen with the second plurality of antibodies.

14. The method of claim 11 wherein the first sample is a purified sample having a reduced concentration of HAP compared to a concentration of HAP present in an unpurified sample.

15. The method of claim 11 wherein the first sample is an unpurified sample.

16. A system for preparing a specimen for analysis, comprising:
a first plurality of antibodies raised to a first sample, the first sample comprising a mixture of HAP and LAP, the first plurality of antibodies attached to a first support.

17. The system of claim 16 further comprising a second plurality of antibodies raised to a second sample, the second sample comprising a mixture of HAP and LAP, the second plurality of antibodies attached to a second support.

18. The system of claim 16 wherein the first sample is a sample purified according to the method of claim 1.

19. The system of claim 16 wherein the first sample is an unpurified sample.

20. The system of claim 17 wherein the first and second supports comprise a material independently selected from the group consisting of: a natural or synthetic polymer, resin, silicate, and a combination thereof.

21. The system of claim 17 wherein the first and second supports comprise a material independently selected from the group consisting of: an agarose; a cellulose, illustratively including a carboxymethyl cellulose; a cellulose acetate; a dextran; a divinylbenzene; a methacrylate; a polymethacrylate; glass; a ceramic; a paper; a metal; a metalloid; a nitrocellulose; or a nylon; a polyacryloylmorpholide; a polyamide; a poly(tetrafluoroethylene); a polyethylene; a polypropylene; poly(4-methylbutene); a poly(ethylene terephthalate); a polyformaldehyde; a polyacrylamide; a polystyrene; a polyethylene glycol; a rayon; a poly(vinyl butyrate); a polyvinylidene difluoride (PVDF); a resin; a silicone; a silicate; and a combination thereof.

22. The system of claim 17 wherein the first and second supports are in a form independently selected from the group consisting of: a membrane, a surface, a bead, a fine particulate, a gel, a matrix, and a combination thereof.

23. The system of claim 16 further comprising a container for the plurality of antibodies attached to a first support.

24. The system of claim 17 further comprising a container for the plurality of antibodies attached to a second support.

25. The system of claim 17 wherein the plurality of antibodies attached to a first support and the plurality of antibodies attached to a second support are present together in a container.

26. A composition comprising:
a purified sample of protein containing material enriched in LAP and having a reduced concentration of HAP compared to an unpurified sample.
